(19) United States
(12) Patent Application Publication
(10) Pub. No.: US 2020/0196885 A1
(43) Pub. Date: Jun. 25, 2020

(54) CATHETER-BASED IDENTIFICATION OF CARDIAC REGIONS

(71) Applicant: Affera, Inc., Watertown, MA (US)

(72) Inventors: Doron Harlev, Watertown, MA (US); Paul B. Hultz, Watertown, MA (US); Geoffrey Peter Wright, Watertown, MA (US)

(21) Appl. No.: 16/633,138

(22) PCT Filed: Aug. 29, 2018
(86) PCT No.: PCT/US2018/048460

§ 371 Date: Jan. 22, 2020

Related U.S. Application Data
(60) Provisional application No. 62/552,019, filed on Aug. 30, 2017.

(51) Int. Cl. A61B 5/042; A61B 5/06; A61B 5/00; A61B 18/14; A61B 5/021; A61B 34/20

(52) U.S. Cl. CPC A61B 5/042; A61B 5/063; A61B 5/062; A61B 5/742; A61B 2018/00351; A61B 18/1492; A61B 5/02116; A61B 34/20; A61B 5/065

(57) ABSTRACT

Devices, systems, and methods directed to detecting positions of a catheter relative to cardiac regions of a patient are disclosed herein. In some implementations, an ablation system can include a cardiac catheter and a catheter interface unit in communication with one or more electrodes and/or sensors on the cardiac catheter. The devices, systems, and methods of the present disclosure can identify regions of the heart based on one or more signals from the respective one or more electrodes and/or sensors. In these and other implementations, the devices, system, and methods can display visual indicia based on an identified cardiac region.

### CROSS-REFERENCE TO RELATED APPLICATION(S)

The present application claims priority to U.S. Provisional Patent Application No. 62/552,019, filed Aug. 30, 2017, the disclosure of which is incorporated herein by reference in its entirety.

### TECHNICAL FIELD

The present technology is generally related to catheter-based identification of cardiac regions and related systems and methods.

### BACKGROUND

In certain cardiac procedures, knowledge of the position of a catheter in the heart is useful for effective treatment, diagnosis, or both. An example of such a cardiac procedure is the termination of certain arrhythmias in the heart through the use of radio frequency (“RF”) ablation. Direct visualization of the heart chamber, however, is often unavailable, incomplete, and/or impractical in cardiac procedures.

To at least partially overcome limitations associated with visualization of the heart chamber, three-dimensional models of the heart chamber are formed prior to or during the procedure and used to guide catheter positioning. Such three-dimensional models are often formed based on measurements of catheter position in the heart and are, therefore, typically subject to constraints associated with imprecise knowledge of the position of the catheter in the heart. Thus, there exists a need for more accurately detecting the position of a catheter in the heart for, among other things, more efficient formation of accurate three-dimensional models useful for guiding catheter positioning in cardiac procedures.

### BRIEF DESCRIPTION OF THE DRAWINGS

The disclosure will be understood more fully from the detailed description given below and from the accompanying drawings of various implementations of the disclosure. The drawings, however, should not be taken to limit the disclosure to the specific implementations, but are for explanation and understanding only.

FIG. 1 is a schematic representation of a system configured in accordance with various implementations of the present technology during a medical procedure on a patient's heart.

FIG. 2 is a perspective view of a catheter of the system shown in FIG. 1 and configured in accordance with various implementations of the present technology.

FIG. 3 is a schematic representation of a tip section of the catheter shown in FIG. 2 in a cardiac chamber and configured in accordance with various implementations of the present technology.

FIG. 4 is a schematic representation of visited locations of the catheter shown in FIG. 2 in the cardiac chamber of FIG. 3 during a build phase of a three-dimensional model of the cardiac chamber in accordance with various implementations of the present technology.

FIG. 5 is a schematic depiction of a projection of a three-dimensional data structure and a continuous surface of the anatomic structure projected to a graphical user interface of the ablation system shown in FIG. 1 in accordance with various implementations of the present technology.

FIGS. 6A-6D are a time progression of electrograms obtained as the tip section of the catheter shown in FIG. 2 is moved in the heart of a swine model in accordance with various implementations of the present technology.

FIGS. 7A-7D are a time progression of electrocardiograms obtained from electrodes substantially fixed to skin of the swine model, with the time progression of the electrocardiograms shown in FIGS. 7A-7D synchronized to the time progression of the electrograms shown in FIGS. 6A-7D in accordance with various implementations of the present technology.

FIG. 8 is a flow chart of a method of displaying visual indicia on a representation of a patient's heart in accordance with various implementations of the present technology.

FIG. 9 is a flow chart of a method of displaying a three-dimensional data structure and/or a representation of a patient's heart in accordance with implementations of the present technology.

FIG. 10 is a flow chart of a method of modifying a graphical user interface in accordance with various implementations of the present technology.

FIG. 11 is a schematic representation of a tip section of the catheter shown in FIG. 2 shown in a cardiac chamber and a coronary sinus catheter positioned in a coronary sinus in accordance with various implementations of the present technology.

### DETAILED DESCRIPTION

### A. Overview

The present disclosure is generally directed to devices, systems, and methods of detecting the position of a catheter relative to cardiac regions of a patient and/or to devices, systems, and methods of identifying one or more cardiac regions corresponding to locations of a catheter moving in the heart of patient. More specifically, the devices, systems, and methods of the present disclosure can identify regions of the heart based on signal(s) from respective one or more sensors supported on a catheter as the catheter is moved within the heart of a patient. Visual indicia corresponding to an identified cardiac region can be displayed on a representation of the patient's heart on a graphical user interface used by a physician as a reference for positioning the catheter in the heart of the patient. The visual indicia can be useful for parsing the graphical representation of the patient's heart into constituent regions. For example, the visual indicia can be used to delineate a valve from one or both of an atrium and a ventricle in the graphical representation of the patient's heart. Further, or instead, the visual indicia can be useful for guiding the physician in moving the catheter in a particular cardiac region. That is, the visual indicia can, in certain instances, provide the physician with an indication that the catheter has moved (e.g., unintentionally) beyond a specific cardiac region. Still further or in the alternative, the identification of a cardiac region can be used to pause location data acquisition while the catheter has moved beyond a specific cardiac region. In such instances, the visual indicia can be used to provide a visual cue to the physician that acquisition of location data (e.g., data used to form the graphical representation of the patient's heart) has been paused during an excursion by the catheter outside of a specific cardiac region. Similarly, the visual indicia can also, or instead, be used to provide a visual cue to the physician that acquisition of location data has resumed upon return of the catheter to a specific cardiac region.

As compared to systems and methods that do not offer such identification of regions of the heart as the catheter is moved within the heart, the devices, systems, and methods of the present disclosure can, for example, reduce the likelihood of unnecessary mapping, treatment, or both of other regions of the heart. Further, or instead, as again compared to systems that do not offer identification of regions of the heart as the catheter is moved within the heart, the devices, systems, and methods of the present disclosure can aid in visualization of the catheter location by representing anatomical landmarks (e.g., a valve) on a user interface. Still further or instead, by identifying regions of the heart as the catheter is moved within the heart, the devices, systems, and methods of the present disclosure can be useful in reducing complexity of a user interface presented to the physician, such as by presenting the physician with only those user interface options relevant to the identified cardiac region. Such a reduction in complexity of the user interface can, in certain instances, reduce the need for the physician to rely on dedicated personnel to manipulate a visualization of the heart during a procedure.

For the sake of clarity of explanation, the devices, systems and methods of the present disclosure are described with respect to medical procedures associated with an ablation catheter used to deliver RF ablation to cardiac tissue in the course of treating certain types of arrhythmias. It should be appreciated, however, that, unless otherwise specified or made clear from the context, the systems and methods of the present disclosure can be used for any of various different medical procedures performed on a cardiac chamber of a patient, in which direct visual access to the medical procedure is impractical and/or is improved by identification of one or more cardiac regions as a catheter is moved in a patient's heart. Thus, for example, the devices, systems, and methods of the present disclosure can be used to facilitate visualization of a catheter inserted into a cardiac chamber as part of a medical treatment associated with diagnosis, treatment, or both of a cardiac condition.

As used herein, the term “physician” shall be understood to include any type of medical personnel who may be performing or assisting a medical procedure and, thus, is inclusive of a doctor, a nurse, a medical technician, other similar personnel, and any combination thereof. Additionally, or alternatively, as used herein, the term “medical procedure” shall be understood to include any manner and form of diagnosis, treatment, or both, inclusive of any preparation activities associated with such diagnosis, treatment, or both, unless a more specific type of medical procedure is identified or made clear from the context. Thus, for example, the term “medical procedure” shall be understood to be inclusive of any manner and form of movement or positioning of a medical device, such as a catheter, in or relative to a cardiac chamber.

As used herein, the term “patient” shall be understood to include any mammal, including a human, upon which a medical procedure is being performed.

As used herein, unless otherwise specified or made clear from the context, the term “cardiac region” shall be understood to include one or more of an atrium, a ventricle, and a valve of a heart of a patient and, further or instead, shall be understood to include veins and arteries of the patient (e.g., the coronary sinus, the inferior and superior vena cava, pulmonary veins, pulmonary arteries, and the aorta).

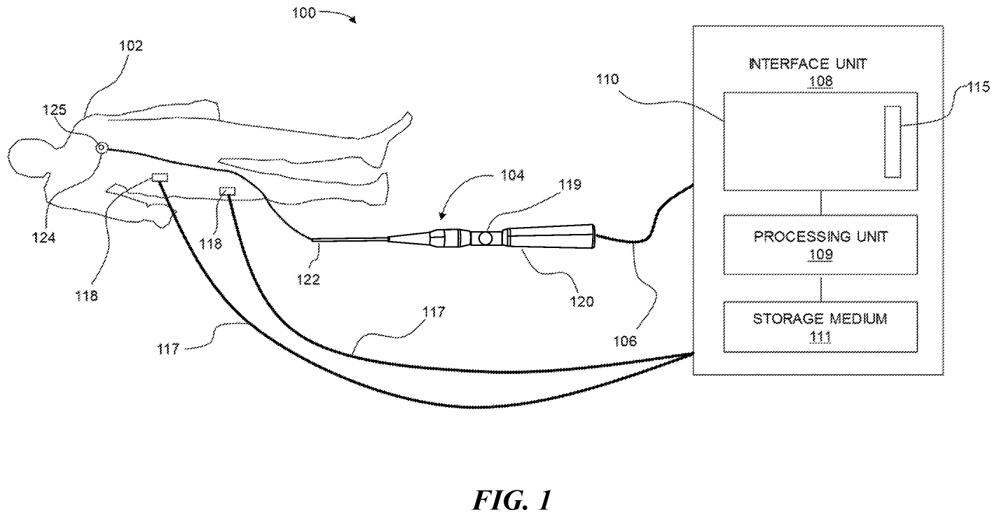

Certain details are set forth in the following description and in FIGS. 1-11 to provide a thorough understandings of various implementations of the disclosure. Other details describing well-known structures and systems often associated with ablation catheters and associated systems and methods, however, are not set forth below to avoid unnecessarily obscuring the description of various implementations of the disclosure.

Many of the details, dimensions, angles, and other features shown in FIGS. 1-11 are merely illustrative of particular implementations of the disclosure. Accordingly, other implementations can have other details, dimensions, angles, and features without departing from the spirit or scope of the present disclosure. In addition, those of ordinary skill in the art will appreciate that further implementations of the disclosure can be practiced without several of the details described below.

### B. Selected Implementations of Catheter-Based Identification of Cardiac Regions and Related Systems and Methods

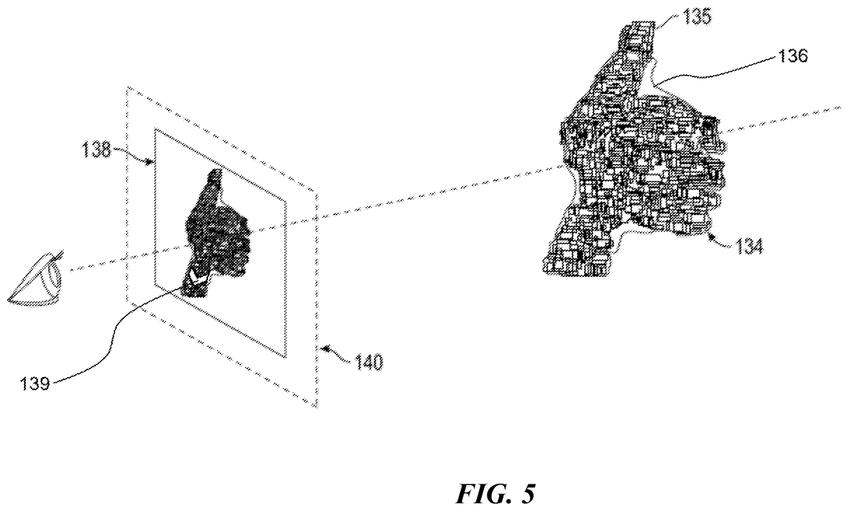

FIG. 1 is a schematic representation of a system 100 configured in accordance with various implementations of the present technology during a medical procedure performed in a cardiac chamber of a patient 102. The system 100 can include a catheter 104 including a tip section 124, and at least one first electrode 125 disposed along the tip section 124. The at least one first electrode 125 can be in electrical communication, via an extension cable 106, with an interface unit 108. The system 100 can further (or instead) include second electrodes or body surface electrodes 118 securable in a substantially fixed position on skin of the patient 102 and in electrical communication with the interface unit 108 via cables 117. The interface unit 108 can include a processing unit 109 (e.g., one or more processors), a graphical user interface 110, and a storage medium 111. The graphical user interface 110 and the storage medium 111 can be in electrical communication (e.g., wired communication, wireless communication, or both) with the processing unit 109. The graphical user interface 110 can include a viewing window 138 (FIG. 5) on which, as described in greater detail below, a representation of at least a portion of the heart of the patient 102 can be displayed. Based on signals associated with the first electrodes 125 and the second electrodes 118, as also described in greater detail below, the processing unit 109 can identify a cardiac region corresponding to a respective one or more locations of the tip section 124 of the catheter 104. As still further described in greater detail below, visual indicia based on the identified cardiac region can be displayed on the representation of the heart of the patient 102 on the graphical user interface 110 to facilitate efficient formation of an accurate model of at least a portion of the heart of the patient 102 and/or to facilitate accurate positioning of the tip section 124 of the catheter 104, or a separate catheter, for the purpose of one or more of diagnosis and treatment of a target area of the heart of the patient 102.

In use, the catheter 104 can be moved within the cardiac chamber (e.g., as part of a medical procedure), and the processing unit 109 can receive a plurality of locations of the catheter 104 in the cardiac chamber during one or more of a build phase and a treatment phase. The build phase can include a portion of a medical procedure in which a portion of the catheter 104 is moved within one or more cardiac cavities to gather anatomical and electrophysiological information related to one or more cardiac chambers. As described in greater detail below, the processing unit 109 can construct a data structure (e.g., a data structure including a three-dimensional data structure) including a representation of locations, within the cardiac chamber, visited by the catheter 104 during the build phase. The data structure can form a basis for a continuous surface displayed on the graphical user interface 110 and representing a blood-tissue boundary in the cardiac chamber. In the treatment phase, the continuous surface displayed on the graphical user interface 110, along with the visual indicia corresponding to one or more identified cardiac regions, can be used as a basis for positioning the catheter 104, or a separate catheter, in a specific position in a cardiac chamber for the delivery of treatment (e.g., delivery of RF ablation energy) to target tissue.

As the catheter 104 is moved within the cardiac chamber during any one or more of the build phase and the treatment phase, the catheter 104 can slip out of the cardiac chamber through a heart valve as the catheter 104 is moved through different locations. In the build phase, such inadvertent positioning of the catheter 104 can result in collection of extraneous location data which, in turn, can result in errors in the graphical representation (e.g., in a continuous surface) of the cardiac chamber to be displayed on the graphical user interface 110. In the treatment phase, such inadvertent positioning of the catheter 104 can increase the time associated with positioning the catheter 104 to deliver treatment to an intended target within a cardiac region.

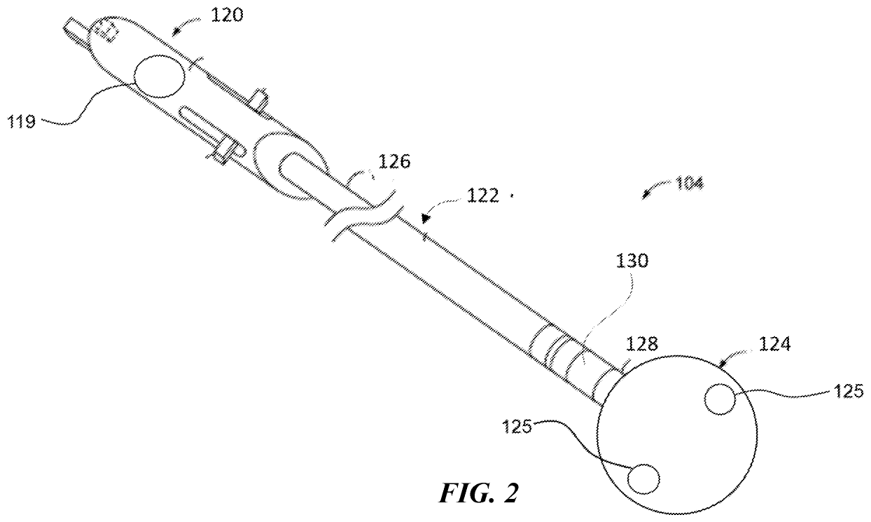

Referring now to FIGS. 1-2, the system 100 is expected to mitigate the impact of inadvertent positioning of the catheter 104. That is, in the build phase, the system 100 can pause data collection upon determining that the tip section 124 of the catheter 104 has moved beyond a given cardiac region. Collection of data can resume upon determining that the tip section 124 of the catheter 104 has returned to a given cardiac region. In the treatment phase, the system 100 can provide a physician with an indication of whether the tip section 124 is in a given cardiac region, such as a cardiac region associated with the intended treatment.

In general, the catheter 104 can be any of various different catheters known in the art for insertion into a cardiac chamber for the purpose of diagnosis, treatment, or both. For example, the catheter can include a handle 120, a shaft 122, and the tip section 124. The shaft 122 can include a proximal portion 126 secured to the handle 120, and a distal portion 128 coupled to the tip section 124.

The one or more first electrodes 125 can be disposed in any of various different orientations relative to the tip section 124, unless otherwise specified or made clear from the context. For example, at least one of the one or more first electrodes 125 can be disposed along an outer surface of the tip section 124 such that the one or more first electrodes 125, so supported, can come into direct contact with cardiac tissue as the tip section 124 is moved within a cardiac chamber. Additionally, or alternatively, at least one of the one or more first electrodes 125 can be disposed away from an outer surface of the tip section 124 such that the one or more first electrodes 125, so supported, do not come into direct contact with cardiac tissue as the tip section 124 is moved within a cardiac chamber.

Each first electrode 125 can detect electrical activity in an area of the heart local to the respective first electrode 125. The detected electrical activity can form a basis for an electrogram associated with an electrode pair that includes the respective first electrode 125. As used herein, the term “electrogram” shall be understood to include an intracardiac electrogram, unless otherwise specified or made clear from the context.

In general, each first electrode 125 can be arranged such that electrical activity detected between an electrode pair that includes the respective first electrode 125 can form the basis of unipolar electrograms, a bipolar electrograms, or other types of electrical signals known in the art. Each first electrode 125 can form an electrode pair with two or more additional electrodes. For example, in implementations in which the one or more first electrodes 125 includes six electrodes, each first electrode 125 can form an electrode pair with each of the other electrodes. Each electrode pair can form the basis for an electrogram.

An electrogram formed by electrical signals received from each respective electrode pair can be generated through any of various different methods. In general, an electrogram associated with a respective electrode pair can be based on a difference between the signals from the electrodes in the pair. Such an electrogram can be filtered or otherwise further processed, for example, to reduce noise and/or to emphasize cardiac electrical activity.

The catheter 104 can further (or instead) include a magnetic position sensor 130 along the distal portion 128 of the shaft 122. It should be appreciated that the magnetic position sensor 130 can be any of various magnetic position sensors well known in the art and can be positioned at any point along the distal portion 128. The magnetic position sensor 130 can, for example, include one or more coils that detect signals emanating from magnetic field generators. One or more coils for determining position with five or six degrees of freedom can be used. Additionally, or alternatively, multiple coils or groups of coils can be placed at different locations along the distal portion 128 to detect the position of different regions of the distal portion 128.

The magnetic field detected by the magnetic position sensor 130 can be used to determine the position of the distal portion 128 of the catheter shaft 122 according to one or more methods commonly known in the art such as, for example, methods based on using a sensor, such as the magnetic position sensor 130, to sense magnetic fields indicative of the position of the magnetic position sensor 130 and using a look-up table to determine a location of the magnetic position sensor 130. Accordingly, because the tip section 124 is coupled to the distal portion 128 of the shaft 122 in a known, fixed relationship to the magnetic position sensor 130, the magnetic position sensor 130 also provides the location of the tip section 124.

While the location of the tip section 124 is described as being determined based on magnetic position sensing, electrical signal feedback, other position sensing methods, or combinations thereof can additionally or alternatively be used. For example, the location of the tip section 124 can be additionally, or alternatively, based on impedance, ultrasound, and/or imaging (e.g., real time MRI or fluoroscopy). Thus, more generally, the location of the tip section 124 at visited positions within the cardiac chamber can be based on one or more location signals generated based on one or more sensors carried on or near the tip section 124, sensors separate from the tip section 124, and combinations thereof.

The catheter 104 can include a remote input device 119 in communication (e.g., wired communication, wireless communication or both) with the interface unit 108. The remote input device 119 can be operated by the physician, from within a sterile field, to navigate, select, or otherwise interact with user interface options 115 displayed on the graphical user interface 110. In certain implementations, the user interface options 115 can reflect a current state of the system. That is, the user interface options 115 can present the physician with relevant input options, given a current state of a medical procedure. As compared to menu structures that require manual navigation, dynamic variation of the user interface options 115 can, for example, reduce the amount of time and attention required by the physician to navigate and select a desired input option.

Referring now to FIGS. 1-4, the tip section 124 of the catheter 104 can be moved in a cardiac chamber 132 in the build phase (e.g., prior to application of the treatment phase). If the tip section 124 of the catheter 104 is movable in blood in the cardiac chamber 132 and obstructed only by a surface 133 of the cardiac chamber 132, the known positions of the tip section 124 of the catheter 104 can be taken together to provide an indication of the size and shape of a volume defined by the cardiac chamber 132 and can form a basis for a three-dimensional data structure corresponding to the volume defined by the cardiac chamber 132.

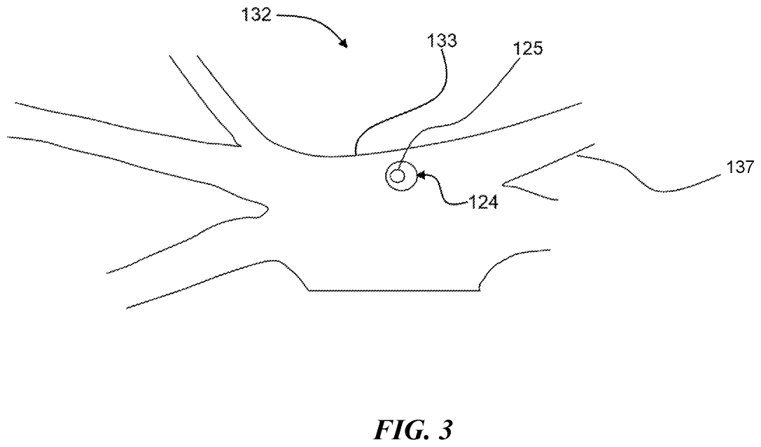

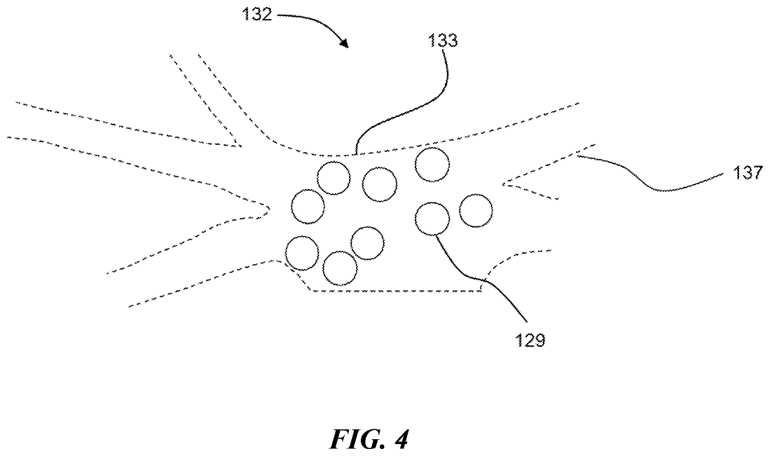

FIG. 4 is a schematic representation of locations 129 visited by the tip section 124 of the catheter 104 (FIGS. 2 and 3) in the cardiac chamber 132 during the build phase. Collectively, the locations 129 can be included in a three-dimensional data structure 134 (FIG. 5) that forms a basis of a graphical representation of the cardiac chamber 132. It should be appreciated, however, that inadvertent excursions of the tip section 124 outside of the cardiac chamber 132 can skew the three-dimensional data structure 134 corresponding to the volume defined by the cardiac chamber 132. For example, movement of the tip section 124 into an anatomic lumen 137, such as a vein, can result in including positions associated with the anatomic lumen 137 into the three-dimensional data structure 134. The inclusion of at least some of these positions can be undesirable. For example, it can be undesirable to include positions in the anatomic lumen 137 that are far removed from (e.g., by a predetermined threshold distance) the electrically active portions of the cardiac chamber 132, as such far removed positions may not be relevant to the diagnosis and treatment of a particular underlying condition. Inclusion of positions that are not relevant to the diagnosis and treatment of the underlying condition can interfere with the display of more clinically-relevant information.

FIG. 5 is a schematic representation of a three-dimensional data structure 134 and a continuous surface 136 projected onto the viewing window 138 of an image plane 140 of the graphical user interface 110 (FIG. 1). While the three-dimensional data structure 134 and the continuous surface 136 can both be projected onto the viewing window 138, it should be understood that the three-dimensional data structure 134 and the continuous surface 136 can be individually projected to the viewing window 138. For example, it can be desirable to project both the three-dimensional data structure 134 and the continuous surface 136 onto the viewing window 138 during the build phase to facilitate editing the three-dimensional data structure 134 and, thus, facilitate editing of the continuous surface 136 according to any one or more of the various different methods described herein. Additionally, or alternatively, it can be desirable to project only the continuous surface 136 (e.g., by making the three-dimensional data structure 134 at least partially translucent) onto the viewing window 138 while the catheter 104 (FIG. 3) is being used to apply a treatment to a cardiac chamber (e.g., the cardiac chamber 132 in FIG. 3).

Referring now to FIGS. 1-5, the three-dimensional data structure 134 can include, for example, a three-dimensional grid of voxels 135. Each voxel 135 can be a discrete element of volume corresponding to an analogous volume in the cardiac chamber 132. Together, the voxels 135 can form the three-dimensional data structure 134 which, more generally, should be understood to be a three-dimensional notional space. Thus, as the tip section 124 of the catheter 104 visits the locations 129 in the cardiac chamber 132, the corresponding one of the voxels 135 can be flagged or otherwise indicated as “visited.” The continuous surface 136 can be formed along a boundary of the voxels 135 of the three-dimensional data structure 134 indicated as “visited.” More specifically, the continuous surface 136 can be extracted from the three-dimensional data structure 134 according to any one or more known computational algorithms for extracting a three-dimensional surface of an object. Examples of such algorithms include one or more of a “marching cubes” algorithm, a “ball-pivoting” algorithm, and a “power crust” algorithm.

The three-dimensional data structure 134 and the continuous surface 136 can be stored, for example, on the storage medium 111, along with instructions executable by the processing unit 109 to display the three-dimensional data structure 134, the continuous surface 136, or both, on the graphical user interface 110, as described in greater detail below. The instructions stored on the storage medium 111 and executable by the processing unit 109 to display one or both of the three-dimensional data structure 134 and the continuous surface 136 can be, for example, an application built using Visualization Toolkit, an open-source 3D computer graphics toolkit, available at www.vtk.org.

The graphical user interface 110 can be two-dimensional (e.g., a screen of a computer monitor) such that the image plane 140 can correspond to a plane of the two-dimensional display of the graphical user interface 110, and the viewing window 138 can correspond to a field of view of the two-dimensional display of the graphical user interface 110. Accordingly, the image formed by projecting one or both of the three-dimensional data structure 134 and the continuous surface 136 onto the viewing window 138 can be displayed on the graphical user interface 110. As described in greater detail below, visual indicia 139 of a cardiac region corresponding to a location of the tip section 124 can be shown on the image formed by projecting the three-dimensional data structure 134, the continuous surface 136, or both onto the viewing window 138 of the graphical user interface 110. Displaying the visual indicia 139 on the graphical user interface 110 can be useful, for example, for providing a physician with guidance with respect to positioning the tip section 124 as part of a medical procedure.

In certain implementations, the visual indicia 139 can include one or more words and/or symbols displayed on a portion of the viewing window 138 away from the projection of the three-dimensional data structure, the continuous surface 136, or both. For example, the visual indicia 139 can display the word “ATRIUM” when the position of the tip section 124 corresponds to an atrium of the heart. Similarly, the visual indicia 139 can display the word “VENTRICLE” when the position of the tip section 124 corresponds to a ventricle of the heart. Likewise, the visual indicia 139 can display the word “VALVE” when the position of the tip section 124 corresponds to a valve of the heart. It should be understood that these specific labels are provided here by way of example, and not limitation, and various different types of labels can be used as visual indicia 139 to appropriately alert the physician. It should be further understood that, as the tip section 124 moves from one cardiac region to another (e.g., from an atrium to a valve), the visual indicia 139 can change accordingly. Further, in some implementations, the graphical user interface 110 can provide the physician with an audible alert (e.g., one or more beeps) indicative of a move from one cardiac region to another.

In some implementations, the visual indicia 139 can include a tag disposed along the three-dimensional data structure 134, the continuous surface 136, or both. For example, the tag can identify the position of a valve of on a graphical representation of the heart represented by the three-dimensional data structure 134, the continuous surface 136, or both. In certain instances, the position of the tip section 124 can also be represented on the graphical representation of the heart and, thus, the position of the tip section 124 relative to the tag can be readily understood by the physician through the projection of the three-dimensional data structure 134, the continuous surface 136, or both onto the viewing window 138 of the graphical user interface 110. As a specific example, in instances in which the visual indicia 139 includes a tag indicative of the position of a valve of the heart, the visual indicia 139 can be used by the physician as a useful anatomic landmark, relative to which the physician can deliver ablation energy or other local treatment to cardiac tissue.

During the build phase, to the extent the locations 129 correspond to excursions by the tip section 124 beyond a given cardiac region (such as the cardiac chamber 132 in the illustrated example), the locations 129 along the excursion can be excluded from the three-dimensional data structure 134. For example, collection of data for the three-dimensional data structure 134 can be paused as the tip section 124 moves along the locations 129 corresponding to the excursion from the given cardiac region. Similarly, data collection for the three-dimensional data structure 134 can resume as the tip section 124 moves along the locations within the given cardiac region. Additionally, or alternatively, data can be collected for the three-dimensional data structure 134 without interruption during an excursion, and the locations 129 corresponding to the excursion can be flagged as corresponding to an excursion. The data flagged as corresponding to an excursion can be, for example, excluded or otherwise deemphasized from subsequent display on the graphical user interface 110.

During one or more of the build phase and the treatment phase, the locations 129 corresponding to excursions by the tip section 124 beyond a given cardiac region can be represented by visual indicia on the graphical user interface. For example, an indication of a cardiac region corresponding to a current location 129 of the tip section 124 can be displayed on the graphical user interface. Continuing with this example, a change in the visual indicia can alert the physician that the tip section 124 has undergone an excursion into a cardiac region different from the cardiac region corresponding to the locations 129 at one or more previous time-steps. Additionally, or alternatively, other types of alerts (e.g., audible alerts) can be used to provide feedback to the physician regarding an excursion. In certain implementations, as described in greater detail below, an approaching excursion from a given cardiac region can be detected and, thus, for example, the physician can be alerted (e.g., via visual indicia on the graphical user interface 110) to an approaching excursion before such an excursion occurs.

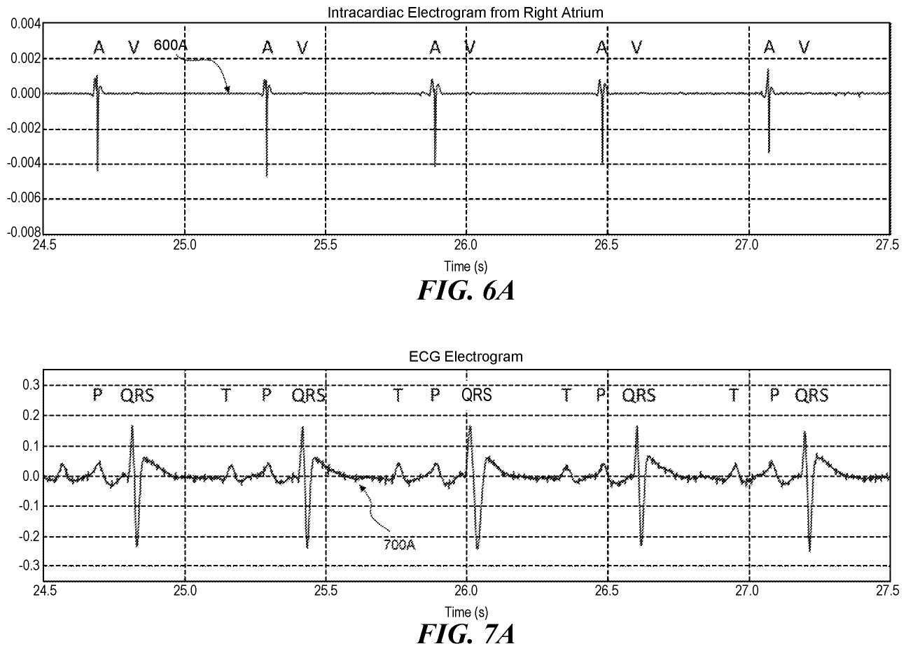

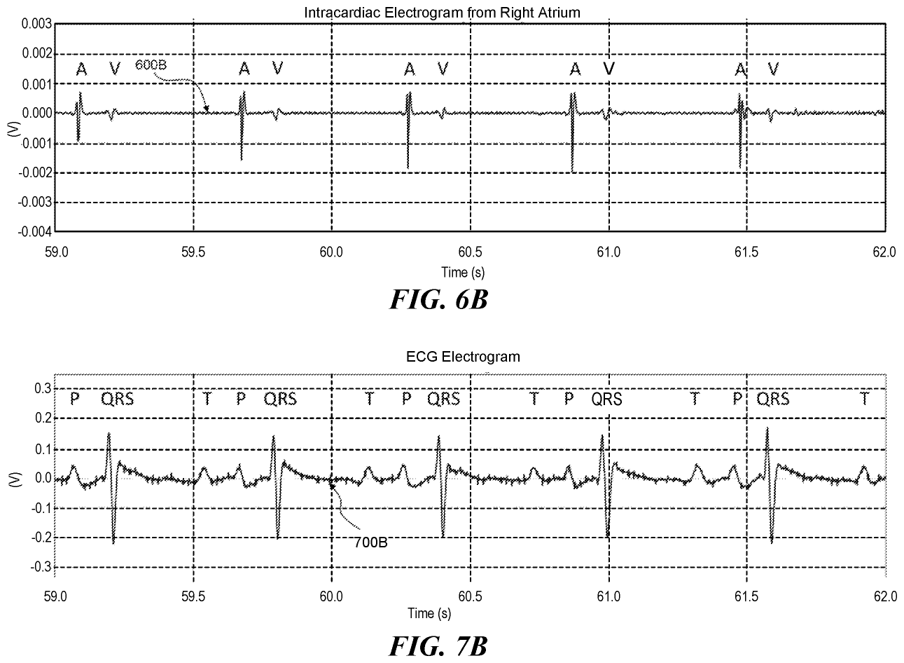

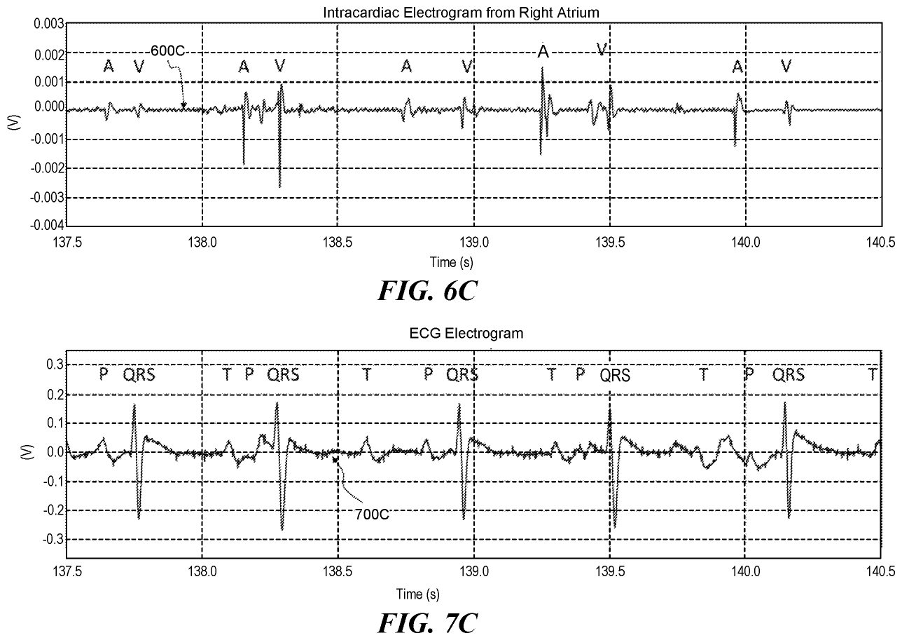

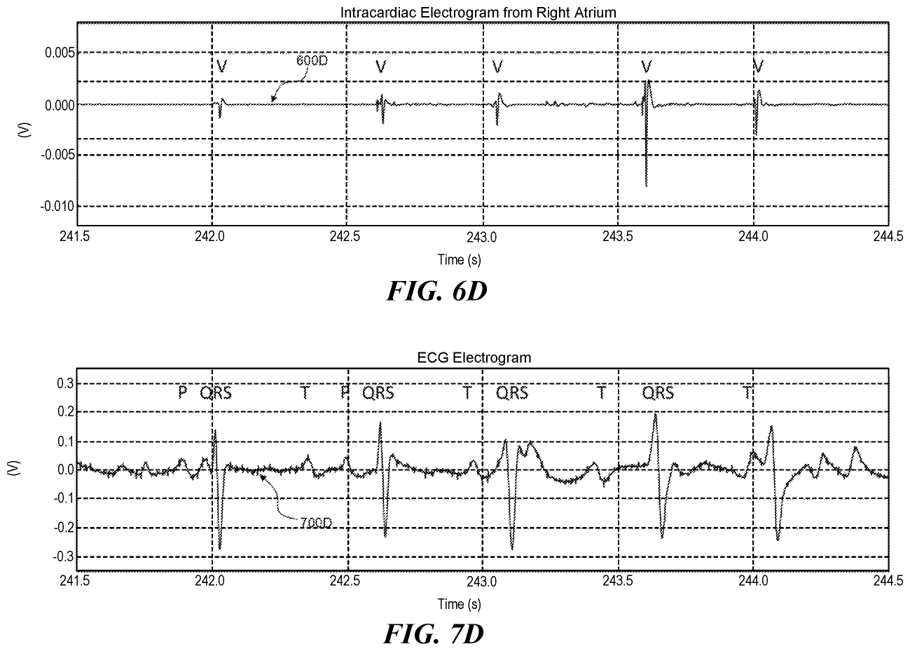

FIGS. 6A-6D illustrate a sequence of electrograms measured in the heart of a swine model using the system 100 (FIG. 1). More specifically, each electrogram in FIGS. 6A-6D is measured between electrodes (e.g., the one or more first electrodes 125 in FIGS. 1-3) on a catheter tip (e.g., the tip section 124 in FIGS. 1-3) moved in the right atrium and right ventricle of the swine model. Each electrogram represents a location of the catheter tip in the heart of the swine model. In particular, the sequence shown in FIGS. 6A-D shows changes in the electrograms as the catheter tip is moved from the right atrium (FIG. 6A) of the heart of the swine model and toward the inside of a valve leading to a ventricle (FIG. 6D) of the heart of the swine model during a right atrial map. For the sake of clarity of explanation, the electrograms shown in FIGS. 6A-6D are measured as the voltage between two of the one or more electrodes 125 and are bandpass filtered between 30 Hz and 300 Hz.

FIGS. 7A-7D illustrate a sequence of electrocardiograms measured between substantially fixed electrodes (e.g., the second electrodes 118 in FIG. 1) on skin of the swine model as the electrodes on the catheter tip were moved in the heart of the swine model to obtain the data shown in FIGS. 6A-6D. Each electrocardiogram shows P-waves “P”, QRS-complexes “QRS,” and T-waves “T” for the timeframe represented in the respective electrocardiogram. The sequences shown in FIGS. 6A-6D and FIGS. 7A-7D are synchronized such that the time associated with the electrogram shown in FIG. 6A corresponds to the time associated with the electrocardiogram shown in FIG. 7A, the time associated with the electrogram shown in FIG. 6B corresponds to the time associated with the electrocardiogram shown in FIG. 7B, and so on for the remainder of each respective sequence.

Referring now to FIGS. 6A-6D and 7A-7D together, identification of a cardiac region corresponding to the location of the catheter tip at a given point in time can be based on the respective electrogram and the respective electrocardiogram at the given point in time. That is, because deflections in an electrocardiogram represent well-known signatures of electrical activity (e.g., depolarization and repolarization) of regions of a beating heart, the deflections in an electrocardiogram can be compared to temporally similar deflections in an electrogram to identify a cardiac region. For example, deflections in the electrograms shown in FIGS. 6A-6D can be matched to one or more of a P-wave representative of atrial depolarization, a T-wave representative of ventricular repolarization, and a QRS complex representative of ventricular depolarization in the electrocardiograms shown in FIGS. 7A-7D. Examples of such comparisons are set forth below with respect to the sequence of catheter tip movement represented in the electrograms shown in FIGS. 6A-6D.

Referring now to FIGS. 6A and 7A together, an electrogram 600A and an electrocardiogram 700A are shown over a time window. The electrogram 600A includes a plurality of deflections “A” occurring at a substantially periodic interval and a plurality of deflections “V” occurring at a substantially periodic interval. In particular, the timing of the deflections “A” in the electrogram 600A corresponds substantially to the timing of P-waves “P” shown in the electrocardiogram 700A, and the plurality of deflections “V” correspond to the QRS-complex “QRS” shown in the electrocardiogram 700A. Given such correspondence of timing, the relative size of the deflections “A” and the deflections “V” in the electrogram 600A indicate that atrial depolarization is the predominant electrical activity detected in the electrogram 600A. That is, the electrical activity detected in the electrogram 600A includes features of electrical activity in an atrium of the heart of the swine model, with relatively little electrical activity from a ventricle of the heart of the swine model. Accordingly, electrogram 600A indicates that the catheter tip position corresponding to the electrogram 600A is in an atrium of the heart of the swine model. As described in greater detail below, as the catheter tip position moves closer to the ventricle, the amount of ventricular electrical activity detected relative to the amount of atrial electrical activity detected increases. Thus, it should be appreciated that the ratio of the amplitude of atrial electrical activity to the amplitude of ventricular electrical activity detected in a given electrogram can provide an indication of the region of the heart in which the electrogram was measured.

Referring now to FIGS. 6B and 7B together, an electrogram 600B and an electrocardiogram 700B are shown over a time window subsequent to the time window shown in FIGS. 6A and 7A. The electrogram 600B is similar to the electrogram 600A (FIG. 6A) in that the electrogram 600B includes the plurality of deflections “A” associated with atrial electrical activity (e.g., the P-wave “P” in FIG. 7B) and the plurality of deflections “V” associated with ventricular electrical activity (e.g., the QRS-complex “QRS” in FIG. 7B). However, as compared to a comparable ratio in FIG. 6A, the ratio of the amplitude of the deflections “V” to the amplitude of the deflections “A” in FIG. 6B is larger. Thus, as compared to the catheter tip position associated with FIG. 6A, the catheter tip position associated with FIG. 6B should be understood to be closer to the ventricle.

In general, the catheter tip must pass through a valve as the catheter tip moves between the atrium and the ventricle. Thus, in certain implementations, the ratio of the amplitude of the deflections “A” to the magnitude of the deflections “V” in an electrogram can be useful for providing an indication of the position of a valve. For example, a predetermined ratio of the amplitude of the deflections “A” to the magnitude of the deflections “V” in an electrogram can be indicative of the position of a valve between an atrium and a ventricle. For example, the predetermined ratio of the amplitude of the deflections “A” to the magnitude of the deflections “V” can be about 1:1. Additionally, or alternatively, the predetermined ratio of the amplitude of the deflections “A” to the magnitude of the deflections “V” can be about 1:5 which can be useful for identifying a boundary spaced a sufficient distance from the valve. Further, or instead, the predetermined ratio of the amplitude of the deflections “A” to the amplitude of the deflections “V” can be input by a physician. In certain instances, a boundary associated with the valve can be a specified distance from locations corresponding to the predetermined ratio of the amplitude of the deflections “A” to the amplitude of the deflections “V.” For example, the locations forming an isosurface corresponding to the predetermined ratio of the amplitude of the deflections “A” to the amplitude of the deflections “V” can be identified, and a boundary can be established relative to such an isosurface. As a more specific example, relative to the isosurface corresponding to the predetermined ratio, the boundary can be set a specified distance in a direction away from the valve to decrease the likelihood of improperly identifying locations as corresponding to a valve.

Referring now to FIGS. 6C and 7C together, an electrogram 600C and an electrocardiogram 700C are shown in a time window subsequent to the time window shown in FIGS. 6B and 7B. The electrogram 600C is similar to the electrograms 600A (FIG. 6A) and 600B (FIG. 6B) in that the electrogram 600C includes the plurality of deflections “A” associated with atrial electrical activity (e.g., the P-wave “P” in FIG. 7C) and the plurality of deflections “V” associated with ventricular electrical activity (e.g., the QRS-complex “QRS” in FIG. 7C). It should be appreciated, however, that the amplitude of some of the deflections “V” in the electrogram 600C are larger than the comparable deflections “V” in FIGS. 6A and 6B and are comparable in magnitude to the amplitude of the deflections “A” in the electrogram 600C. Thus, as compared to the positions of the catheter tip associated with the electrograms 600A and 600B (FIGS. 6A-B), the position of the catheter tip associated with the electrogram 600C (FIG. 6C) should be understood to be closer to a ventricle of the heart of the swine model. Additionally, or alternatively, based on a ratio of the amplitude of the deflections “A” to the amplitude of the deflections “V” in the electrogram 600C, the position of the catheter tip associated with the electrogram 600C can be identified as corresponding to a valve of the heart of the swine model. The identification of the valve in this position of the catheter tip can be useful, for example, for tagging or otherwise visually representing the presence of the valve on a graphical representation of the heart. Such a tag or other visual representation can facilitate, for example, delivery of ablation energy along a perivalvular path.

Referring now to FIGS. 6D and 7D together, an electrogram 600D and an electrocardiogram 700D are shown in a time window subsequent to the time window shown in FIGS. 6C and 7C. As compared to the electrogram 600C (FIG. 6C), it should be appreciated that the electrogram 600D includes a plurality of deflections “V” associated with ventricular electrical activity (e.g., the QRS-complex “QRS” in FIG. 7D), but is substantially flat elsewhere along the electrogram 600D. In particular, the electrogram 600D does not include deflections associated with atrial electrical activity. Accordingly, the position of the catheter tip associated with the electrogram 600D should be understood to be in a ventricle of the heart of the swine model.

Referring now to FIGS. 1, 2, and 5 together, the computer executable instructions stored on the storage medium 111 (FIG. 1) can cause the processing unit 109 (FIG. 1) to identify a cardiac region associated with the position of the tip section 124 according to one or more of the following methods. Unless otherwise indicated, each of the following methods can be implemented using the system 100 (FIG. 1) and/or one or more components thereof. In other implementations, however, other suitable systems may be utilized to perform the disclosed methods.

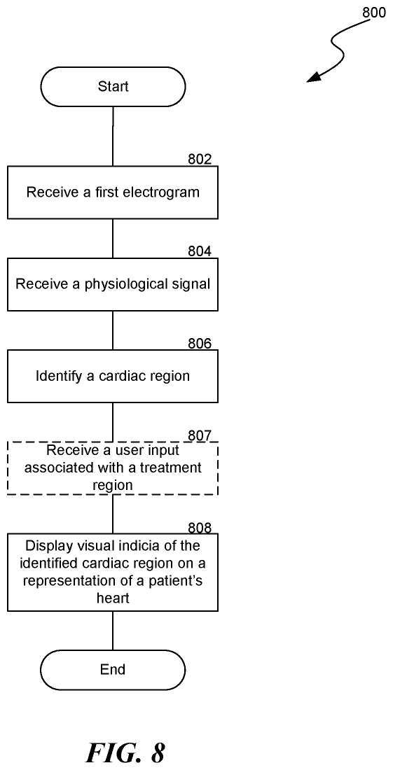

FIG. 8 is a flowchart of a method 800 of displaying visual indicia on a representation of a patient's heart. At block 802, the method 800 can include receiving a first electrogram associated with locations of one or more first electrodes of a catheter in a patient's heart. The method further includes receiving a physiological signal associated with sensors at respective substantially fixed locations relative to the patient's heart at block 804, and identifying a cardiac region corresponding to a respective one or more of the locations associated with the first electrogram at block 806. Based on the identified cardiac region corresponding to the respective one or more locations of the one or more first electrodes, the method continues at block 808 with displaying visual indicia on a graphical user interface. As described in greater detail below, the identifying step at block 806 can be based on the first electrogram and the physiological signal. In general, the physiological signal can provide context (e.g., as described above with respect to FIGS. 6A-6D and FIGS. 7A-7D) useful for interpreting features of the first electrogram as being indicative of a cardiac region. At block 808, displaying the visual indicia on a graphical user interface can be useful for providing visual cues to the physician as the physician moves the catheter in the heart of the patient. As compared to performing a medical procedure without the benefit of such visual cues, it should be appreciated that displaying the visual indicia at block 808 can facilitate positioning the one or more first electrodes at a desired location in the heart for diagnosis, treatment, or both.

Referring again to block 802, receiving the first electrogram can include receiving respective electrical signals from the one or more first electrodes of the catheter. In general, the electrical signals from the one or more first electrodes of the catheter can be based a voltage, a current, an impedance, or a combination thereof between a pair of electrodes that includes a first electrode. The first electrogram can be based on a difference between electrical signals measured between one or more electrode pairs and, thus, can be any one or more of the various different types of electrograms described herein. For example, the first electrogram can be unipolar, bipolar, or other types of electrical signals known in the art.

The electrical signals forming the basis for the first electrogram are time-varying signals. These electrical signals can be processed according to any one or more different known signal processing techniques useful for reducing noise in a signal. As an example, the electrical signals can be band-pass filtered. In general, processing the electrical signals to reduce noise can facilitate identifying characteristic electrical activity in the first electrogram. That is, processing the electrical signals can increase the likelihood of properly identifying deflections characteristic of atrial electrical activity, ventricular electrical activity, or both.

In certain implementations, receiving the first electrogram at block 802 can include associating the first electrogram with a location of the one or more first electrodes in the heart of the patient at the time the electrical signals for a given first electrogram are acquired. Such association between the first electrogram and a location of the one or more first electrodes can facilitate, for example, tagging the location in a graphical representation of the heart as a particular type of cardiac region (e.g., as determined from the first electrogram according to any one or more of the methods described herein). More generally, as the first electrodes move within the heart of the patient, each location can be tagged based on features of the respective first electrogram associated with the location.

Referring again to block 804, receiving the physiological signal associated with the second electrodes can include receiving the physiological signal over a timeframe at least partially overlapping a timeframe of the first electrogram. The temporal coordination of the physiological signal with the first electrogram can be useful, for example, for comparing the first electrogram to the physiological signal according to any one or more of the various different methods described herein and, in particular, according to any one or more of the methods described below.

The physiological signal can be any of various different types of physiological signals associated with the heart of the patient and suitable for providing context for analysis of the first electrogram. Thus, for example, the physiological signal can include a signal received from one or more sensors secured in a substantially fixed location on a body surface of a patient. As a more specific example, the physiological signal can include an electrocardiogram based on surface electrodes (e.g., the second electrodes 118 shown in FIG. 1) secured in substantially fixed locations on a body surface (e.g., skin) of the patient. Known signatures of electrical activity in the electrocardiogram can be used to analyze the first electrogram according to any one or more of the methods described herein.

In general, the physiological signal can be a time-varying signal. Thus, to facilitate comparison of the physiological signal to the first electrogram, the physiological signal can be processed according to any one or more of various different signal processing techniques known in the art. For example, the physiological signal can be band-pass filtered. The electrogram and the physiological signal can be filtered differently. As an example, to account for differences in high-frequency components and high-frequency noise, the electrograms can be filtered with a higher upper cutoff frequency than the physiological signal. It should be appreciated, however, that first electrogram and the physiological signal can be processed according to the same type of filter in certain implementations.

Referring to block 806, identifying the cardiac region corresponding to the respective one or more locations of the one or more first electrodes in the patient's heart can include determining whether the respective one or more of the locations of the one or more first electrodes corresponds to at least one predetermined type of cardiac region. As a specific example, identifying the cardiac region can include determining whether the respective location of the one or more first electrodes corresponds to one or more of an atrium, a ventricle, and a valve. Identification of these cardiac regions—or, more generally, distinguishing these cardiac regions from one another—can be useful, for example, for providing a physician with guidance with respect to placement of the one or more first electrodes.

In certain instances, identifying the cardiac region at block 806 can include determining a probability that a given location of the one or more first electrodes is in a given cardiac region. For example, locations can be assigned different probabilities of being in a given cardiac region based on the first electrogram associated with a given location of the one or more first electrodes and, more specifically, based on a ratio of an amplitude of a deflection associated with the cardiac region to an amplitude of a deflection associated with a different cardiac region. In certain implementations, the probability associated with each location can be stored in a three-dimensional data structure. As described in greater detail below, probability information from multiple measurements in nearby locations can be aggregated to reduce errors in the stored data (e.g., through spatial averaging or smoothing).

In implementations of the method 800 in which identifying the cardiac region (block 806) includes determining a probability associated with a given location, the probability information associated with each location can be useful in the formation of a representation of a continuous surface of the heart. Such a surface representation can be based on any one or more of various different numerical algorithms known in the art to extract the continuous surface from the three-dimensional data structure. As an example, a “marching cubes” algorithm can be useful for slicing through the three-dimensional data structure along an isosurface of the probability value. Additionally, or alternatively, tags or an isocontour can be placed along an extracted surface at points corresponding to a given probability value. As a more specific example, in the case of an isocontour, the extracted surface can be cut to remove an unwanted region from the graphical representation of the heart (e.g., to create an opening corresponding to a valve).

In some instances, identifying the cardiac region (block 806) can include providing an indication of proximity of a present location of the one or more first electrodes to a valve. For example, based on the first electrogram, identifying the cardiac region can include identifying whether a location in one or both of the atrium or the ventricle is near the valve. Further, or instead, identifying the cardiac region can include comparing relative proximity of two different locations (e.g., a current location and one or more previous locations) of the one or more first electrodes to provide an indication of whether the one or more first electrodes are moving toward the valve or away from the valve.

Identifying the cardiac region (block 806) can include comparing portions of the first electrogram to one another based on timing of one or more features of the physiological signal. For example, the physiological signal can include features associated with known electrical activity in the heart of the patient, and the timing of deflections in the first electrogram can be compared to the known electrical activity in the physiological signal. Deflections in the first electrogram that are temporally aligned with deflections of known electrical activity in the physiological signal can be associated with the known electrical activity. For example, as described with respect to FIGS. 6A-6D and FIGS. 7A-7D, deflections in the first electrogram that are temporally aligned with a P-wave in the physiological signal can be associated with detected atrial activity. Further, or instead, deflections in the first electrogram that are temporally aligned with a QRS complex in the physiological signal can be associated with detected ventricular activity.

In general, identifying the cardiac region (block 806) can include comparing a first amplitude of a first portion of the first electrogram to a second amplitude of a second portion of the first electrogram. In some implementations, identifying the type of cardiac region can be based on one or more predetermined thresholds of a ratio of the first amplitude to the second amplitude. As an example, the cardiac region corresponding to a respective one or more of the locations of the one or more first electrodes can be identified as an atrium of the patient's heart based on whether the ratio of the first amplitude to the second amplitude is greater than a first threshold. Additionally, or alternatively, the cardiac region corresponding to a respective one or more of the locations of the one or more first electrodes can be identified as a ventricle of the patient's heart based on whether the ratio of the first amplitude to the second amplitude is less than a second threshold. Still further, or instead, the cardiac region corresponding to a respective one or more of the locations of the one or more first electrodes can be identified as a valve of the patient's heart based on whether the ratio of the first amplitude to the second amplitude is between the first threshold and the second threshold. FIGS. 6A-6D and 7A-7D and the corresponding description of those figures provides still more specific examples of the use a ratio of deflections in the first electrogram as a basis for distinguishing locations associated with an atrium, a ventricle, and a valve from one another. Thus, it should be understood that the first amplitude of the first portion of the first electrogram can correspond to timing of a P-wave in the physiological signal and, further or instead, the second amplitude of the second portion of the first electrogram can correspond to timing of a QRS complex in the physiological signal.

Parsing the first electrogram into the first portion and the second portion can be based on a temporal window defined along the physiological signal. An R-wave window is an example of a temporal window that can be defined in the physiological signal and useful for parsing the first electrogram. For example, the first portion of the electrogram can correspond to timing outside of the R-wave window and between R-wave peaks of successive heartbeats in the physiological signal, and the second portion of the electrogram can correspond to timing within the R-wave window of the physiological signal. As used herein, an R-wave window should be understood to include a temporal window at least partially defined relative to an R-wave detected in the physiological signal. Thus, as an example, an R-wave window can be a temporal window of a fixed duration (e.g., about 200 ms) about a detected R-wave. Further or instead, an R-wave window can be a temporal window having a duration that is a multiple (e.g., about 2X) of a duration of a QRS complex of the physiological signal. Still further or instead, an R-wave window can be a temporal window defined relative to both a P-wave and an R-wave. The R-wave is typically the most easily identifiable waveform in the physiological signal, particularly in instances in which the physiological signal is an electrocardiogram. Accordingly, the timing associated with an R-wave window along the physiological signal can facilitate robust and repeatable parsing of the first electrogram.

Identifying the cardiac region (block 806) can also be based on the first electrogram and the physiological signal over a plurality of heartbeats. As compared to making a determination over a single heartbeat, identifying the cardiac region based on information associated with a plurality of heartbeats is expected to reduce the likelihood of interference from spurious data. Accordingly, identifying the cardiac region based on signals received over a plurality of heartbeats is expected to facilitate robust identification of the cardiac region over a variety of conditions. For example, identifying the cardiac region based on information associated with a plurality of heartbeats can be useful for assigning a probability that the given location is in a predetermined cardiac region. As a more specific example, a location identified as corresponding to an atrium over a plurality of heartbeats can be associated with a high probability of corresponding to an atrium while a location identified as variously corresponding to an atrium and a valve over a plurality of heartbeats can be associated with a lower probability of corresponding to an atrium. Thus, in general, the identification of the cardiac region based on information associated with a plurality of heartbeats is expected to reduce the influence of outlying data. Further, or instead, a probability associated with a given location can be aggregated with probability information from similar measurements in nearby locations to reduce the influence of outlying data. Examples of such aggregation can include, among other things, spatial averaging or smoothing across nearby locations.

Displaying visual indicia on a graphical user interface (block 808) can include providing the visual indicia on a representation of a patient's heart displayed on the graphical user interface. As an example, the visual indicia can be displayed on one or more of a three-dimensional data structure and a continuous surface representative of a surface of the patient's heart (e.g., the three-dimensional data structure 134 and the continuous surface 136 in FIG. 5). Further, or instead, displaying visual indicia (block 808) according to the method 800 can include any one or more of various different display techniques useful for providing a physician with improved visualization of the identified cardiac region. As used herein, improved visualization should be understood to include any one or more of various different display techniques useful for distinguishing the identified cardiac region from any one or more other cardiac regions. Further, or instead, it should be appreciated that displaying the visual indicia can be associated with any one or more of various different displays on the graphical user interface during one or both of a build phase and a treatment phase.

In certain implementations, displaying the visual indicia (block 808) can include displaying only those portions of the representation of the patient's heart corresponding to the at least one predetermined type of cardiac region. For example, in instances in which the predetermined type of cardiac region is an atrium, displaying the visual indicia can include displaying only those portions of the representation of the patient's heart corresponding to the atrium. Thus, continuing with this example, as the one or more first electrodes are inadvertently moved into a ventricle, the locations of the one or more first electrodes associated with the ventricle can be excluded from the displayed visual indicia. In analogous examples, the predetermined type of cardiac region can be one or more of a ventricle and a valve.

Further, or instead, the predetermined type of cardiac region can include more than one type of cardiac region such that a particular type of cardiac region can be excluded from the displayed visual indicia. As an example, the predetermined type of cardiac region can be an atrium and a valve such that displaying the visual indicia includes displaying those portions of the representation of the patient's heart corresponding to the atrium and the valve, thus excluding only those portions of the representation of the patient's heart corresponding to the ventricle. Additionally, or alternatively, displaying the visual indicia can be based on removing a valve from the representation of the patient's heart, as is often useful for providing a physician with visualization of a cardiac region adjacent to the valve. More generally, any combination of one or more types of cardiac regions can be selected such that any one or more types of cardiac regions can be included or excluded from the displayed visual indicia according to the needs of a particular use-case (e.g., a build phase and a treatment phase), physician preference, or a combination thereof.

In some implementations, displaying the visual indicia (block 808) can include modifying the visual indicia on the representation of the patient's heart as the one or more first electrodes move from a first cardiac region to a second cardiac region, with the first cardiac region being different from the second cardiac region. For example, modifying the visual indicia can include changing a color of the representation of the patient's heart at the location of the one or more first electrodes, with the color providing a readily perceptible signal to the physician that the one or more of the first electrodes have migrated from an intended cardiac region. Additionally, or alternatively, modifying the visual indicia can include displaying an alert (e.g., in the form of a symbol, text, or a combination thereof) to draw the physician's attention to deviation from an intended cardiac region.

In certain implementations, displaying the visual indicia (block 808) based on the identified cardiac region corresponding to the respective one or more of the locations of the one or more first electrodes can include coloring at least a portion of the representation of the patient's heart based on the identified cardiac region. For example, one or more predetermined cardiac regions can be displayed as a color differing from the other predetermined cardiac regions. More specifically, coloring at least a portion of the representation of the patient's heart can include coloring locations corresponding to a valve as a color differing from one or both of the atrium and the ventricle. In general, displaying the cardiac regions as different colors can provide the physician with readily perceptible cues regarding the location of the one or more first electrodes relative to a given cardiac region. Additionally, or alternatively, in instances in which each location of the one or more first electrodes are associated with a probability of being in a given cardiac region, the color of the location can correspond to a gradient reflecting the probability. It should be appreciated that such a gradient can result in a gradual transition in color on the graphical user interface.

In some implementations, displaying the visual indicia (block 808) can include adjusting opacity of at least a portion of the representation of the patient's heart based on the identified cardiac region. For example, on the representation of the patient's heart displayed on the graphical user interface, a predetermined type of cardiac region can be displayed as less opaque than one or more other types of cardiac regions. By displaying the predetermined type of cardiac region with less opacity on the graphical user interface, the physician's attention can be directed to the predetermined type of cardiac region.

In certain implementations, displaying the visual indicia (block 808) can include displaying a contour along a portion of the representation of the patient's heart. That is, the contour can be representative of locations between an atrium and a ventricle and corresponding, therefore, to a valve. The contour can be useful, for example, for providing the physician with a useful landmark for positioning the one or more first electrodes, such as during a treatment phase of a treatment desirably applied relative to the valve (e.g., a treatment, such as RF ablation, that is advantageously applied near the valve).

In certain implementations, the method 800 can further include receiving a user input associated with a selection of a treatment region at block 807. As used herein, the treatment region can include one or more types of cardiac regions and, thus, for example, can include one or more of an atrium, a supraventricular region (e.g., a region including the atrium and/or one or more other anatomic structures situated above the ventricles), and a ventricle. The user input associated with the selection of the treatment region can be made in a variety of ways, including through interaction with a user interface on a catheter interface unit, interaction with a user interface on the catheter (e.g., on a handle of the catheter), voice commands, hand gestures, or a combination thereof. As an example, a list of one or more types of cardiac regions can be displayed on a graphical user interface (e.g., the graphical user interface 110), and the physician can select one or more of the types of cardiac regions through the use of an input device in communication with the graphical user interface.

The physician can, for example, set up the system for a particular procedure or for a portion of a procedure, and the method 800 is expected to provide the physician with feedback regarding whether an identified cardiac region at a given location of the catheter (block 806) corresponds to the target treatment region associated with such a procedure or portion of a procedure. Such feedback can be provided to the physician in real-time or substantially in real-time such that the physician can adjust the position of the catheter as necessary in instances in which the catheter inadvertently moves from the treatment region to another cardiac region. As an example, displaying the visual indicia (block 808) can include displaying an indication of whether the identified cardiac region (block 806) corresponds to the received user input associated with the selection of the treatment region (block 807). The visual indicia, therefore, can provide the physician with a readily perceivable indication of whether the catheter is in an intended cardiac region, allowing the physician to adjust the position of the catheter as necessary.

While the visual indicia can be represented as changes to one or more of a three-dimensional data structure and a continuous surface based on the three-dimensional data structure, other types of visual indicia are additionally or alternatively possible. For example, the visual indicia can include a tag. Continuing with this example, the tag can be associated with a given location of the one or more first electrodes and displayed on or near the three-dimensional data structure, the continuous surface, or both. As an example, the tag can represent a location of a valve or, similarly, another location useful for delivery of a treatment. As an additional or alternative example, displaying visual indicia (block 808) can include changing a representation of the catheter on the graphical user interface. Such a change to the representation of the catheter can include any of various changes suitable for providing a readily perceptible visual cue to the physician as the physician's attention is directed to the representation of the catheter during a procedure. As an example, changing the representation of the catheter on the graphical user interface can include changing a color of the representation of the catheter.

While the visual indicia can be used to provide an indication to the physician, it should be appreciated that, more generally, an audible alert, a haptic alert, or other types of alerts can be additionally or alternatively used to provide feedback to a physician.

Although the steps of the method 800 are discussed and/or illustrated in a particular order, the method 800 shown in FIG. 8 is not so limited. In other implementations, the method 800 can be performed in a different order. In these and other implementations, any of the steps of the method 800 can be performed before, during, and/or after any of the other steps of the method 800. Moreover, a person of ordinary skill in the relevant art will readily recognize that the illustrated method 800 can be altered and still remain within these and other implementations of the present technology. For example, one or more steps of the method 800 illustrated in FIG. 8 can be omitted and/or repeated in some implementations.

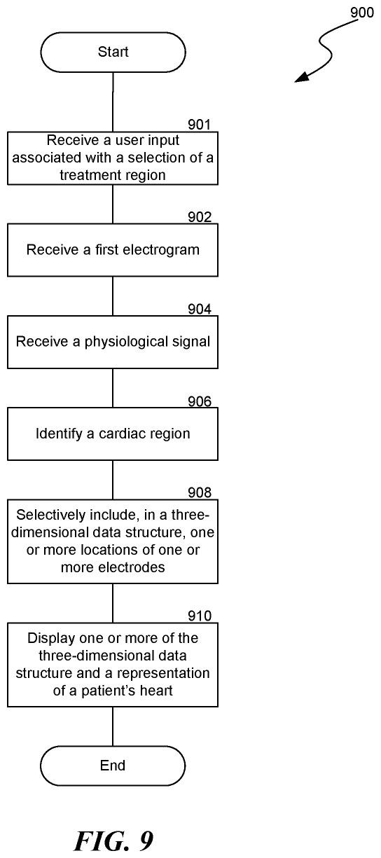

FIG. 9 is a flow chart of a method 900 of displaying one or more of a three-dimensional data structure and a representation of a patient's heart (e.g., the three-dimensional data structure 134 and the continuous surface 136 shown in FIG. 5). In general, the method 900 can be useful during a build phase. For example, as one or more first electrodes of a catheter are moved in the heart of the patient to gather location data useful for forming a three-dimensional data structure and, in certain instances, a continuous surface representative of a surface of a patient's heart, the method 900 can be used to exclude certain extraneous locations (e.g., locations associated with unintended migration of the one or more first electrodes away from a cardiac region of interest). The exclusion of extraneous locations from the three-dimensional data structure during the build phase can be useful for efficiently and accurately forming the three-dimensional data structure and, thus, can reduce the overall time associated with a medical procedure.

Beginning at block 901, the method 900 can include receiving a user input associated with a selection of a treatment region in a patient's heart. Referring to block 902, the method 900 continues with receiving a first electrogram associated with one or more first electrodes of a catheter at a plurality of first locations in a patient's heart, and at block 904 the method 900 includes receiving a physiological signal different from the first electrogram. The method 900 further includes identifying a cardiac region corresponding to a respective one or more of the locations of the one or more first electrodes in the patient's heart (block 906), selectively including the respective one or more locations of the one or more first electrodes in a three-dimensional data structure (block 908), and displaying one or more of the three-dimensional data structure and a representation of the patient's heart (block 910). Unless otherwise specified or made clear from the context, receiving the user input associated with the selection of a treatment region in the patient's heart (block 901), receiving the first electrogram (block 902), and receiving the physiological signal (block 904) should be understood to be analogous to the respective receiving the user input associated with the selection of a treatment region (block 807), receiving the first electrogram (block 802), and receiving the physiological signal (block 804) processes described above with respect to FIG. 8. Similarly, identifying the cardiac region at block 906 can be based on the first electrogram and the physiological signal and, unless otherwise indicated or made clear from the context, should be understood to be analogous to identifying the cardiac region (block 806) described above with respect to FIG. 8.

In general, selectively including the respective one or more locations of the one or more first electrodes in the three-dimensional data structure (block 908) can be based on whether the identified cardiac region (block 906) corresponds to the treatment region associated with the selection received as the user input (block 901). Thus, in instances in which the treatment region is an atrium, selectively including the respective one or more locations (block 908) can be based on whether the one or more locations are identified as corresponding to an atrium (block 906). Continuing with this example, locations identified as corresponding to an atrium (block 906) can be included in the three-dimensional data structure while locations that are identified (block 906) as not corresponding to an atrium (e.g., corresponding to a valve or a ventricle) can be excluded from the three-dimensional data structure. Analogous examples should be understood to be applicable with respect to the predetermined cardiac region being a valve or a ventricle. More generally, selectively including the respective one or more locations of the one or more first electrodes in the three-dimensional data structure (block 908) can be based on movement of the one or more first electrodes from a first cardiac region to a second cardiac region, with the first cardiac region being different from the second cardiac region.

In some implementations, selectively including the respective one or more locations of the one or more first electrodes in the three-dimensional data structure (block 908) can include adding any one or more of various different types of location information to the three-dimensional data structure. For example, selectively including the respective one or more of the locations of the one or more first electrodes in the three-dimensional data structure (block 908) can include adding the coordinates of the respective one or more of the locations of the one or more first electrodes to the three-dimensional data structure. Further, or instead, at least one representation (e.g., a schematic representation) of one or more portions of the catheter (e.g., the catheter 104 in FIG. 2) can be added to the three-dimensional data structure. For example, in instances in which locations of electrodes on a catheter are identified (block 906) as corresponding to the treatment region associated with the selection received at block 901 as the user input, at least one shape corresponding to the distal portion of the catheter 104 at the one or more locations can be added to the three-dimensional data structure. Continuing with this example, in instances in which none of the locations of electrodes on the catheter 104 are identified (block 906) as corresponding to the treatment region associated with the selection received at block 901 as the user input, the catheter representation can be excluded from the three-dimensional data structure. At block 908, selectively including a representation of one or more portions of the catheter 104 can, further or instead, be based on other rules. For example, the representation of one or more portions of the catheter can be excluded if any of the electrodes on the catheter are identified at block 906 as corresponding to a region different from the treatment region associated with the selection received at block 901 as the user input.

Although the steps of the method 900 are discussed and/or illustrated in a particular order, the method 900 shown in FIG. 9 is not so limited. In other implementations, the method 900 can be performed in a different order. In these and other implementations, any of the steps of the method 900 can be performed before, during, and/or after any of the other steps of the method 900. Moreover, a person of ordinary skill in the relevant art will readily recognize that the illustrated method 900 can be altered and still remain within these and other implementations of the present technology. For example, one or more steps of the method 900 illustrated in FIG. 9 can be omitted and/or repeated in some implementations.

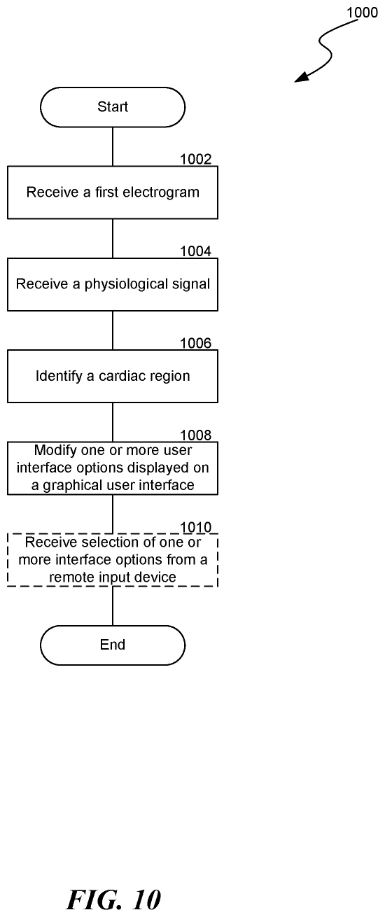

FIG. 10 is a flow chart of a method 1000 of modifying a graphical user interface. The method 1000 can be useful, for example, for presenting the physician with the most relevant user input options based on a cardiac region corresponding to a given location of the one or more first electrodes. For example, as the one or more first electrodes move from an atrium to a ventricle, the method 1000 can advantageously modify user input options presented to the physician such that the user input options are relevant to the cardiac region corresponding to the given location of the one or more first electrodes. Such adaptation of the user input options can be useful, for example, for reducing the amount of attention required by the physician to navigate menu options associated with one or more of a build phase and a treatment phase.

Beginning at block 1002, the method 1000 can include receiving a first electrogram associated with one or more first electrodes of a catheter at first locations in a patient's heart. The method 1000 continues at block 1004 with receiving a physiological signal different from the first electrogram, and at block 1006 with identifying a cardiac region corresponding to a respective one or more of the locations of the one or more first electrodes in the patient's heart. At block 1008, the method 1000 comprises modifying one or more user interface options displayed on a graphical user interface based on the identified cardiac region. Unless otherwise specified or clear from the context, receiving the first electrogram (block 1002) and receiving the physiological signal (block 1004) are analogous to the respective receiving the first electrogram (block 802) and receiving the physiological signal (block 804) described above with respect to FIG. 8. Similarly, identifying the cardiac region (block 1006) can be based on the first electrogram and the physiological signal and, unless otherwise indicated or clear from the context, should be understood to be analogous to identifying the cardiac region (block 806) described above with respect to FIG. 8.

In certain implementations, modifying one or more user interface options displayed on the graphical user interface (block 1008) can include modifying a state of a state machine displayed on a graphical user interface. As an example, the state of a state machine displayed on a graphical user interface can correspond to one or more states associated with the identified cardiac region (block 1006). Thus, as the identified cardiac region at block 1006 changes through movement of the one or more first electrodes in the heart of the patient, the state of the state machine displayed on the graphical user interface can change accordingly. Such dynamic variation of the state of the state machine can advantageously present the physician with relevant input options associated with the identified cardiac region (block 1006) and, optionally, with respect to a mode of use (e.g., a build phase or a treatment phase).

In certain implementations, the method 1000 can further include receiving, from a remote input device (e.g., the remote input device 119 shown in FIGS. 1 and 2), a selection of the one or more user interface options at block 1010. Because the user interface options displayed on the graphical user interface at any given time can be modified (block 1008) based on the identified cardiac region (block 1006), the physician can select a desired input option using only a few input commands. The use of only a few commands can, in turn, be implemented through a remote input device with a few buttons and, in certain implementations, such a remote input device can be incorporated into the catheter.

Although the steps of the method 1000 are discussed and/or illustrated in a particular order, the method 1000 shown in FIG. 10 is not so limited. In other implementations, the method 1000 can be performed in a different order. In these and other implementations, any of the steps of the method 1000 can be performed before, during, and/or after any of the other steps of the method 1000. Moreover, a person of ordinary skill in the relevant art will readily recognize that the illustrated method 1000 can be altered and still remain within these and other implementations of the present technology. For example, one or more steps of the method 1000 illustrated in FIG. 10 can be omitted and/or repeated in some implementations.

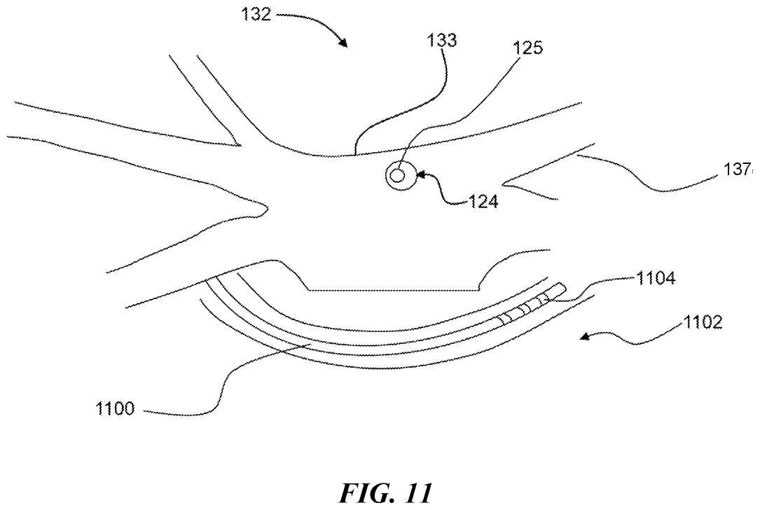

While certain implementations have been described, other implementations are additionally or alternatively possible. For example, while the physiological signal has been described as being an electrocardiogram based on electrodes at substantially fixed locations on skin of the patient, other implementations are additionally, or alternatively, possible. As an example, referring now to FIGS. 1 and 11 together, a coronary sinus catheter 1100 can be positioned in a coronary sinus 1102 of the patient as the tip section 124 is moved within the cardiac chamber 132. Second electrodes or intracardiac reference electrodes 1104 disposed on the coronary sinus catheter 1100 can acquire one or more signals at a substantially fixed location in the coronary sinus of the patient while the at least one first electrode 125 acquires one or more signals associated with an electrogram of the cardiac chamber 132. As used herein, the substantially fixed location of the second electrodes 1104 in the coronary sinus of the patient should be understood to allow for incidental movement of the coronary sinus catheter 1100 during a medical procedure.

The one or more signals acquired by the second electrodes 1104 of the coronary sinus catheter 1100 can form a basis for a second electrogram. In general, unless otherwise indicated or made clear from the context, the second electrogram measured by the second electrodes 1104 on the coronary sinus catheter 1100 can form a basis for any one or more of the physiological signals described herein. As an example, the coronary sinus catheter 1100 can be in electrical communication with the interface unit 108, and the second electrogram measured by the coronary sinus catheter 1100 can form the basis of a physiological signal used, in combination with the first electrogram measured by the at least one first electrode 125, to identify a cardiac region according to any one or more of the methods described herein.

As another example, while physiological signals have been described as being based on signals measured from electrodes in substantially fixed positions relative to the heart of a patient, other physiological signals are additionally or alternatively possible. For example, the physiological signal can be based on a pressure waveform measured by one or more pressure sensors positioned to measure changes in pressure in an anatomic vessel as the heart of the patient beats. For example, the pressure sensors can be pressure sensors disposed in an anatomic vessel of the patient as the heart of the patient beats.

As still another example, while electrograms have been described herein as being compared to physiological signals to determine timing suitable for identifying cardiac regions, the electrograms described herein can be compared to other types of signals to calibrate deflections shown in the electrograms to electrical activity of the heart, and other types of comparisons are additionally or alternatively possible for associating deflections in the electrograms with electrical activity of the heart. For example, a signal based on flow of blood in an anatomic chamber or vessel can be useful for establishing timing suitable for associating deflections in the electrograms to electrical activity of the heart. As one specific example, ultrasound can be used to determine timing associated with the flow of blood in an anatomic chamber or vessel, and this timing can be compared to deflections in the electrograms to identify a cardiac region. As an additional or alternative example, thermal dilution can be used to determine timing associated with the flow of blood in an anatomic vessel, and this timing can be compared to deflections in the electrograms to identify a cardiac region.

Multiple physiological signals can be used, in combination with the first electrogram measured by the first electrode(s) 125 to identify a cardiac region according to any one or more of the methods described herein. For example, multiple signals acquired from second electrodes or body surface electrodes 118 on the skin of the patient can be used together to identify a timing of an R-wave or a QRS complex using detection algorithms known in the art. Additionally or alternatively, the multiple physiological signals can comprise multiple signal types such as, for example, electrogram signals and pressure signals.

The above systems, devices, methods, processes, and the like can be realized in hardware, software, or any combination of these suitable for a particular application. The hardware can include a general-purpose computer and/or dedicated computing device. This includes realization in one or more microprocessors, microcontrollers, embedded microcontrollers, programmable digital signal processors or other programmable devices or processing circuitry, along with internal and/or external memory. This can also, or instead, include one or more application specific integrated circuits, programmable gate arrays, programmable array logic components, or any other device or devices that can be configured to process electronic signals.

It will further be appreciated that a realization of the processes or devices described above can include computer-executable code created using a structured programming language such as C, an object oriented programming language such as C++, or any other high-level or low level programming language (including assembly languages, hardware description languages, and database programming languages and technologies) that can be stored, compiled or interpreted to run on one of the above devices, as well as heterogeneous combinations of processors, processor architectures, or combinations of different hardware and software. In another aspect, the methods can be embodied in systems that perform the steps thereof, and can be distributed across devices in a number of ways. At the same time, processing can be distributed across devices such as the various systems described above, or all of the functionality can be integrated into a dedicated, standalone device or other hardware. In another aspect, means for performing the steps associated with the processes described above can include any of the hardware and/or software described above. All such permutations and combinations are intended to fall within the scope of the present disclosure.

Implementations disclosed herein can include computer program products comprising computer-executable code or computer-usable code that, when executing on one or more computing devices, performs any and/or all of the steps thereof. The code can be stored in a non-transitory fashion in a computer memory, which can be a memory from which the program executes (such as random-access memory (RAM) associated with a processor), or a storage device such as a disk drive, flash memory or any other optical, electromagnetic, magnetic, infrared or other device or combination of devices.

In another aspect, any of the systems and methods described above can be embodied in any suitable transmission or propagation medium carrying computer-executable code and/or any inputs or outputs from same.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y, and Z can include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

### C. Additional Examples

Several aspects of the present technology are set forth in the following examples.

1. A method comprising:
receiving a first electrogram associated with one or more first electrodes of a catheter at locations in a patient's heart;
receiving a physiological signal associated with second electrodes at respective substantially fixed locations relative to the patient's heart;
based on the first electrogram and the physiological signal, identifying a cardiac region corresponding to a respective one or more of the locations of the one or more first electrodes in the patient's heart; and
on a graphical user interface, displaying visual indicia based on the identified cardiac region corresponding to the respective one or more locations of the one or more first electrodes.

2. The method of example 1 wherein displaying the visual indicia includes providing the visual indicia on a representation of the patient's heart displayed on the graphical user interface.

3. The method of example 2 wherein displaying the visual indicia includes coloring at least a portion of the representation of the patient's heart based on the identified cardiac region.

4. The method of any one of examples 2 or 3 wherein displaying the visual indicia based on the identified cardiac region includes adjusting opacity of at least a portion of the representation of the patient's heart based on the identified cardiac region.

5. The method of any one of examples 1-4 wherein displaying the visual indicia includes changing a representation of the catheter on the graphical user interface.

6. The method of any one of examples 1-5 wherein identifying the cardiac region includes determining whether the respective one or more of the locations of the one or more first electrodes corresponds to at least one predetermined type of cardiac region.

7. The method of example 6 wherein displaying the visual indicia includes displaying only those portions of a representation of the patient's heart on the graphical user interface corresponding to the at least one predetermined type of cardiac region.

8. The method of any one of examples 6 and 7 wherein identifying the cardiac region includes determining whether the respective one or more of the locations of the one or more first electrodes corresponds to an atrium of the patient's heart.

9. The method of any one of examples 6-8 wherein identifying the cardiac region includes determining whether the respective one or more of the locations of the one or more first electrodes corresponds to a ventricle of the patient's heart.

10. The method of any one of examples 6-9 wherein identifying the cardiac region includes determining whether the respective one or more of the locations of the one or more first electrodes corresponds to a valve of the patient's heart.

11. The method of any one of examples 1-10 wherein displaying the visual indicia includes modifying the visual indicia as the one or more first electrodes move from a first cardiac region to a second cardiac region, the first cardiac region different from the second cardiac region.

12. The method of any one of examples 1-11 wherein, based on the identified cardiac region corresponding to a valve of the patient's heart, displaying the visual indicia based on the identified cardiac region includes displaying a contour along a portion of a representation of the patient's heart on the graphical user interface.

13. The method of any one of examples 1-12 further comprising receiving a user input associated with a selection of a treatment region in the patient's heart, wherein displaying the visual indicia includes displaying an indication of whether the identified cardiac region corresponds to the selected treatment region.

14. The method of example 13 wherein the selected treatment region is an/the atrium and displaying the visual indicia includes displaying an indication of whether the identified cardiac region corresponds to the atrium.

15. The method of any one of examples 1-14 wherein the visual indicia include a tag.

16. The method of any one of examples 1-15 wherein identifying the cardiac region includes comparing a first portion in the first electrogram to a second portion in the first electrogram, the first portion is different from the second portion, and the comparison of the first portion to the second portion is based on timing of one or more features of the physiological signal.

17. The method of example 16 wherein identifying the cardiac region includes processing one or both of the first electrogram and the physiological signal.

18. The method of example 17 wherein processing one or both of the first electrogram and the physiological signal includes band-pass filtering one or both of the first electrogram and the physiological signal.

19. The method of any one of examples 16-18 wherein identifying the cardiac region includes comparing a first amplitude of a first portion of the first electrogram to a second amplitude of a second portion of the first electrogram.

20. The method of example 19 wherein the first amplitude of the first portion of the first electrogram corresponds to timing of a P-wave in the physiological signal, and the second amplitude of the second portion of the first electrogram corresponds to timing of a QRS complex in the physiological signal.

21. The method of example 20 wherein the second portion of the first electrogram corresponds to timing of an R-wave window of the physiological signal and the first portion of the first electrogram corresponds to timing outside of the R-wave window and between R-wave peaks of successive heartbeats in the physiological signal.

22. The method of example 21 wherein a duration of the R-wave window is about twice as large as a duration of a QRS complex of the physiological signal.

23. The method of any one of examples 19-22 wherein identifying the cardiac region is based on one or more predetermined thresholds of a ratio of the first amplitude to the second amplitude.

24. The method of any one of examples 1-23 wherein identifying the cardiac region corresponding to the respective one or more of the locations of the one or more first electrodes in the patient's heart is based on the first electrogram and the physiological signal over a plurality of heartbeats.

25. The method of any one of examples 1-24 wherein the first electrogram is an electrogram associated with a pair of the one or more first electrodes on the catheter.

26. The method of any one of examples 1-25 wherein the physiological signal includes an electrocardiogram associated with the second electrodes in a substantially fixed position on a body surface of the patient.

27. The method of any one of examples 1-26 wherein the physiological signal includes a second electrogram associated with the second electrodes in a substantially fixed position in an anatomic structure of the patient.

28. The method of example 27 wherein the second electrodes are in a substantially fixed position in a coronary sinus of the patient.

29. A method comprising:
receiving a user input associated with a selection of a treatment region in a patient's heart; receiving a first electrogram associated with one or more first electrodes of a catheter at first locations in the patient's heart;
receiving a physiological signal different from the first electrogram;
based on the first electrogram and the physiological signal, identifying a cardiac region corresponding to a respective one or more of the locations of the one or more first electrodes in the patient's heart; based on whether the identified cardiac region corresponds to the treatment region, selectively including the respective one or more of the locations of the one or more first electrodes in a three-dimensional data structure; and
on a graphical user interface, displaying one or more of the three-dimensional data structure and a representation of the patient's heart, wherein the representation of the patient's heart is based on the three-dimensional data structure.

30. The method of example 29 wherein the treatment region includes an atrium of the patient's heart.

31. The method of example 30 wherein the treatment region includes a supraventricular region.

32. The method of any one of examples 30 or 31 wherein the treatment region includes a ventricle of the patient's heart.

33. The method of any one of examples 30-32 wherein identifying the cardiac region corresponding to the respective one or more of the locations of the one more first electrodes in the patient's heart includes comparing portions in the first electrogram to one another based on timing of one or more features of the physiological signal.

34. The method of example 33 wherein identifying the cardiac region corresponding to the respective one or more of the locations of the one or more first electrodes in the patient's heart includes processing one or both of the first electrogram and the physiological signal.

35. The method of example 34 wherein processing one or both of the first electrogram and the physiological signal includes band-pass filtering one or both of the first electrogram and the physiological signal.

36. The method of any one of examples 29-35 wherein identifying the cardiac region corresponding to the respective one or more of the locations of the one or more first electrodes in the patient's heart includes comparing a first amplitude of a first portion of the first electrogram to a second amplitude of a second portion of the first electrogram.

37. The method of example 36 wherein the first amplitude of the first portion of the first electrogram corresponds to timing of a P-wave in the physiological signal, and the second amplitude of the second portion of the first electrogram corresponds to timing of a QRS complex in the physiological signal.

38. The method of example 37 wherein the second portion of the first electrogram corresponds to timing of an R-wave window of the physiological signal and the first portion of the first electrogram corresponds to timing outside of the R-wave window and between R-wave peaks of successive heartbeats in the physiological signal.

39. The method of example 38 wherein a duration of the R-wave window is about twice as large as a duration of a QRS complex of the physiological signal.

40. The method of any one of examples 36-39 wherein identifying the cardiac region is based on one or more predetermined thresholds of a ratio of the first amplitude to the second amplitude.

41. The method of any one of examples 29-40 wherein identifying the cardiac region corresponding to the respective one or more of the locations of the one or more first electrodes in the patient's heart is based on the first electrogram and the physiological signal over a plurality of heartbeats.

42. The method of any one of examples 29-41 wherein selectively including the respective one or more of the locations of the one or more first electrodes in the three-dimensional data structure is based on movement of the one or more first electrodes from a first cardiac region to a second cardiac region, the first cardiac region different from the second cardiac region.

43. The method of any one of examples 29-42 wherein selectively including the respective one or more of the locations of the one or more first electrodes in the three-dimensional data structure includes adding coordinates of the respective one or more of the locations to the three-dimensional data structure.

44. The method of any one of examples 29-43 wherein selectively including the respective one or more of the locations of the one or more first electrodes in the three-dimensional data structure includes adding at least one representation of one or more portions of the catheter to the three-dimensional data structure.

45. The method of any one of examples 29-44 wherein receiving the physiological signal is associated with second electrodes at respective substantially fixed locations relative to the patient's heart.

46. The method of any one of examples 29-45 wherein the physiological signal includes one or more of a second electrogram and an electrocardiogram.

47. A method comprising:
receiving a first electrogram associated with one or more first electrodes of a catheter at first locations in a patient's heart;
receiving a physiological signal different from the first electrogram;
based on the first electrogram and the physiological signal, identifying a cardiac region corresponding to a respective one or more of the first locations of the one or more first electrodes in the patient's heart; and
based on the identified cardiac region, modifying one or more user interface options displayed on a graphical user interface.

48. The method of example 47 wherein identifying the cardiac region includes determining whether the respective one or more of the first locations of the one or more first electrodes corresponds to at least one predetermined type of cardiac region.

49. The method of example 48 wherein identifying the cardiac region includes determining whether the respective one or more of the first locations of the one or more first electrodes corresponds to an atrium of the patient's heart.

50. The method of any one of examples 48 or 49 wherein identifying the cardiac region includes determining whether the respective one or more of the first locations of the one or more first electrodes corresponds to a ventricle of the patient's heart.

51. The method of any one of examples 48-50 wherein identifying the cardiac region includes determining whether the respective one or more of the first locations of the one or more first electrodes corresponds to a valve of the patient's heart.

52. The method of any one of examples 47-51 wherein identifying the cardiac region corresponding to the respective one or more of the first locations of the one or more first electrodes in the patient's heart includes comparing portions in the first electrogram to one another based on timing of one or more features of the physiological signal.

53. The method of example 52 wherein identifying the cardiac region corresponding to the respective one or more of the first locations of the one or more first electrodes in the patient's heart includes processing one or both of the first electrogram and the physiological signal.

54. The method of example 53 wherein processing one or both of the first electrogram and the physiological signal includes band-pass filtering one or both of the first electrogram and the physiological signal.

55. The method of any one of examples 47-54 wherein identifying the cardiac region corresponding to the respective one or more of the first locations of the one or more first electrodes in the patient's heart includes comparing a first amplitude of a first portion of the first electrogram to a second amplitude of a second portion of the first electrogram.

56. The method of example 55 wherein the first amplitude of the first portion of the first electrogram corresponds to timing of a P-wave in the physiological signal, and the second amplitude of the second portion of the first electrogram corresponds to timing of a QRS complex in the physiological signal.

57. The method of example 56 wherein the second portion of the first electrogram corresponds to timing of an R-wave window of the physiological signal and the first portion of the first electrogram corresponds to timing outside of the R-wave window and between R-wave peaks of successive heartbeats in the physiological signal.

58. The method of example 57 wherein a duration of the R-wave window is about twice as large as a duration of a QRS complex of the physiological signal.

59. The method of any one of examples 55-58 wherein identifying the cardiac region is based on one or more predetermined thresholds of a ratio of the first amplitude to the second amplitude.

60. The method of any one of examples 47-59 wherein identifying the cardiac region corresponding to the respective one or more of the first locations of the one or more first electrodes in the patient's heart is based on the first electrogram and the physiological signal over a plurality of heartbeats.

61. The method of any one of examples 47-60 wherein modifying the one or more user interface options on the graphical user interface includes modifying a state of a state machine displayed on the graphical user interface.

62. The method of any one of examples 47-61 further comprising receiving, from a remote input device, a selection of the one or more user interface options.

63. The method of any one of examples 47-62 wherein the received physiological signal is associated with second electrodes at respective substantially fixed locations relative to the patient's heart.

64. The method of any one of examples 47-63 wherein the physiological signal includes one or more of a second electrogram and an electrocardiogram.

65. A non-transitory, computer-readable storage medium having stored thereon computer executable instructions for causing one or more processors to execute the method of any one or more of examples 1-64.

66. A system comprising:
a cardiac catheter including:
   a shaft having a proximal end portion and a distal end portion, and
   one or more first electrodes, the one or more first electrodes mechanically coupled to the distal end portion of the shaft;
a second catheter including second electrodes; and
a catheter interface unit in electrical communication with the first electrodes and the second electrodes, the catheter interface unit including a graphical user interface, one or more processors, and the non-transitory, computer-readable storage medium of example 65.

67. The system of example 66 wherein the second catheter is a coronary sinus catheter.

68. The system of any one of examples 66 or 67 further comprising a remote input device in electrical communication with the catheter interface unit to select one or more user interface options displayed on the graphical user interface.

69. A system comprising:
a cardiac catheter including:
   a shaft having a proximal end portion and a distal end portion, and
   one or more first electrodes, the one or more first electrodes mechanically coupled to the distal end portion of the shaft;
second electrodes securable in a substantially fixed position on skin of a patient; and
a catheter interface unit in electrical communication with the first electrodes and the second electrodes, the catheter interface unit including a graphical user interface, one or more processors, and the non-transitory, computer-readable storage medium of example 65.

70. The system of example 69 further comprising a remote input device in electrical communication with the catheter interface unit to select one or more user interface options displayed on the graphical user interface.

### D. Conclusion

The above detailed descriptions of implementations of the present technology are not intended to be exhaustive or to limit the technology to the precise form disclosed above. Although specific implementations of, and examples for, the technology are described above for illustrative purposes, various equivalent modifications are possible within the scope of the technology, as those skilled in the relevant art will recognize. For example, while steps are presented in a given order, alternative implementations can perform steps in a different order. Furthermore, the various implementations described herein can also be combined to provide further implementations.

From the foregoing, it will be appreciated that specific implementations of the present technology have been described herein for purposes of illustration, but well-known structures and functions have not been shown or described in detail to avoid unnecessarily obscuring the description of the implementations of the present technology. Where the context permits, singular or plural terms can also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Where the context permits, singular or plural terms can also include the plural or singular term, respectively. Additionally, the terms “comprising,” “including,” “having” and “with” are used throughout to mean including at least the recited feature(s) such that any greater number of the same feature and/or additional types of other features are not precluded. To the extent any materials incorporated herein by reference conflict with the present disclosure, the present disclosure controls.

From the foregoing, it will also be appreciated that various modifications can be made without deviating from the technology. For example, various components of the technology can be further divided into subcomponents, or that various components and functions of the technology can be combined and/or integrated. Furthermore, although advantages associated with certain implementations of the present technology have been described in the context of those implementations, other implementations can also exhibit such advantages, and not all implementations need necessarily exhibit such advantages to fall within the scope of the present technology.

1. A method comprising:
receiving a first electrogram associated with one or more first electrodes of a catheter at locations in a patient's heart;
receiving a physiological signal associated with second electrodes at respective substantially fixed locations relative to the patient's heart;
based on the first electrogram and the physiological signal, identifying a cardiac region corresponding to a respective one or more of the locations of the one or more first electrodes in the patient's heart; and
on a graphical user interface, displaying visual indicia based on the identified cardiac region corresponding to the respective one or more locations of the one or more first electrodes.

2. The method of claim 1 wherein displaying the visual indicia includes providing the visual indicia on a representation of the patient's heart displayed on the graphical user interface.

3. The method of claim 2 wherein displaying the visual indicia includes coloring at least a portion of the representation of the patient's heart based on the identified cardiac region.

4. The method of claim 2, wherein displaying the visual indicia based on the identified cardiac region includes adjusting opacity of at least a portion of the representation of the patient's heart based on the identified cardiac region.

5. The method of claim 1 wherein displaying the visual indicia includes changing a representation of the catheter on the graphical user interface.

6. The method of claim 1 wherein identifying the cardiac region includes determining whether the respective one or more of the locations of the one or more first electrodes corresponds to at least one predetermined type of cardiac region.

7. The method of claim 6 wherein displaying the visual indicia includes displaying only those portions of a representation of the patient's heart on the graphical user interface corresponding to the at least one predetermined type of cardiac region.

8. The method of claim 6 wherein identifying the cardiac region includes determining whether the respective one or more of the locations of the one or more first electrodes corresponds to an atrium of the patient's heart.

9. The method of claim 6 wherein identifying the cardiac region includes determining whether the respective one or more of the locations of the one or more first electrodes corresponds to a ventricle of the patient's heart.

10. The method of claim 6 wherein identifying the cardiac region includes determining whether the respective one or more of the locations of the one or more first electrodes corresponds to a valve of the patient's heart.

11. The method of claim 1 wherein displaying the visual indicia includes modifying the visual indicia as the one or more first electrodes move from a first cardiac region to a second cardiac region, the first cardiac region different from the second cardiac region.

12. The method of claim 1 wherein, based on the identified cardiac region corresponding to a valve of the patient's heart, displaying the visual indicia based on the identified cardiac region includes displaying a contour along a portion of a representation of the patient's heart on the graphical user interface.

13. The method of claim 1 further comprising receiving a user input associated with a selection of a treatment region in the patient's heart, wherein displaying the visual indicia includes displaying an indication of whether the identified cardiac region corresponds to the selected treatment region.

14. The method of claim 13 wherein the selected treatment region is an atrium and displaying the visual indicia includes displaying an indication of whether the identified cardiac region corresponds to the atrium.

15. The method of claim 1 wherein the visual indicia include a tag.

16. The method of claim 1 wherein identifying the cardiac region includes comparing a first portion in the first electrogram to a second portion in the first electrogram, the first portion is different from the second portion, and the comparison of the first portion to the second portion is based on timing of one or more features of the physiological signal.

17. The method of claim 16 wherein identifying the cardiac region includes processing one or both of the first electrogram and the physiological signal.

18. The method of claim 17 wherein processing one or both of the first electrogram and the physiological signal includes band-pass filtering one or both of the first electrogram and the physiological signal.

19. The method of claim 16 wherein identifying the cardiac region includes comparing a first amplitude of a first portion of the first electrogram to a second amplitude of a second portion of the first electrogram.

20. The method of claim 19 wherein the first amplitude of the first portion of the first electrogram corresponds to timing of a P-wave in the physiological signal, and the second amplitude of the second portion of the first electrogram corresponds to timing of a QRS complex in the physiological signal.

21-76. (canceled)
